(19) United States
(12) Patent Application Publication
(10) Pub. No.: US 2015/0141833 A1
(43) Pub. Date: May 21, 2015

(54) CATHETER DEVICE IMPLEMENTING HIGH FREQUENCY, CONTRAST IMAGING ULTRASOUND TRANSDUCER, AND ASSOCIATED METHOD

(71) Applicant: North Carolina State University, Raleigh, NC (US)

(72) Inventors: Paul Dayton, Carrboro, NC (US); Xiaoning Jiang, Cary, NC (US)

(21) Appl. No.: 14/403,767

(22) PCT Filed: May 29, 2013
(86) PCT No.: PCT/US2013/043002

§ 371 Date: Nov. 25, 2014

Related U.S. Application Data
(60) Provisional application No. 61/654,335, filed on Jun. 1, 2012.

(51) Int. Cl. A61B 8/00; A61B 8/08; A61B 8/12

(52) U.S. Cl. CPC A61B 8/445; A61B 8/12; A61B 8/481; A61B 8/0891; A61B 8/4494
USPC 600/458; 600/463

(57) ABSTRACT

An imaging catheter system is provided, including a hollow lumen having a distal portion, and a plurality of first transducer elements arranged in a first array configured to be received within the distal portion of the hollow lumen. Each of the first transducer elements include a micromachined piezocomposite, and the plurality of first transducer elements is configured to operate at an effective operational frequency of greater than about 30 MHz. A plurality of second transducer elements configured to operate at an effective operational frequency of less than about 15 MHz may be arranged in a second array, each of the second transducer elements including a micromachined piezocomposite, and engaged with the first array. The low frequency array may be operated in a transmit mode and the high frequency array may be operated in a receive mode to facilitate contrast imaging.

### BACKGROUND OF THE INVENTION

1. Field of the Invention

The present invention relates to imaging catheter devices and more particularly to a catheter device implementing a high frequency, contrast imaging transducer arrangement, and associated method.

2. Description of Prior Art

Cardiovascular disease (atherogenesis) is the leading cause of cardiovascular mortality and morbidity. Approximately every 25 seconds, an American will have a cardiac event and about every minute an American will die from the cardiac event. Cardiovascular (or heart) disease is the leading cause of mortality in the United States and is a major cause of disability. In 2010, approximately 785,000 Americans experienced a new cardiac event and 470,000 Americans experienced a reoccurring event. Prevention and detection of this disease are critical to the survival rate for a patient. The most common ways to prevent cardiovascular disease are through behavioral changes and routine visits to the patient's primary care physician. This is difficult because the existence of the disease is not always readily apparent as it does not manifest many physical symptoms. A physician can assist a patient to prevent or diagnose a potential cardiac event through their awareness of physical changes to the patient such as shortness of breath, dizziness, fatigue, etc. Regarding detection, the most common methods are the monitoring of blood pressure and cholesterol. By monitoring these two factors, physicians can determine the patient's susceptibility to cardiovascular disease but these are not definitive measurements for the disease.

The most definitive detection method is by monitoring the level of plaque in the patient's veins and arteries, with plaque being composed of cholesterol, fat, calcium, scar tissue and other substances found in the blood. Plaque can clog and block arteries. Currently, physicians can use three methods of determining the amount of plaque in patients' arteries by using either the vascular ultrasound imaging process, angiography, or intravascular ultrasound imaging process. The vascular ultrasound imaging process, also called ultrasound scanning or sonography, involves exposing part of the body to high-frequency sound waves to produce pictures of the inside of the body. Because ultrasound images are captured in real-time, the process can show the blood flowing through blood vessels or arteries. Ultrasound imaging is a noninvasive process that provides pictures of the veins and arteries. A Doppler ultrasound study is normally part of this process which is a technique that evaluates blood flow through the blood vessel. The angiography process is a way to produce X-ray pictures of the inside of blood vessels. Angiography provides a non-invasive process to determine the source of the problem and the extent of damage to the blood vessel segments that are being examined. The intravascular ultrasound imaging process is an invasive procedure, performed along with cardiac catheterization; a miniature sound probe (transducer) on the tip of a coronary catheter is threaded through the coronary arteries and, by using high-frequency sound waves, the process produces detailed images of the interior walls of the arteries. Where angiography shows a two-dimensional silhouette of the interior of the coronary arteries, intravascular ultrasound imaging process shows a cross-section of both the interior, and the layers of the artery wall itself. The intravascular imaging process allows the physician to view the artery from the inside out, making it possible to evaluate the amount of disease present and how it is distributed.

Each of these methods is able to illustrate the presence of plaque in vessels and determine the blood flow within the vessel but none are able to determine how susceptible the plaque is to movement within the vessel or its propensity for growth. The previously described processes can determine the physical characteristics of the plaque, such as the thickness and if it is smooth or un-even surface on the plaque. Imaging resolution, penetration depth into calcified tissue, and tissue classification challenges are obstacles to more comprehensive assessment of the plaque when using the aforementioned processes. These processes also can not detect the internal properties of the plaque such as the blood flow within the plaque and the vulnerability of the plaque to dislodge and cause a blood clot in the patient.

Thus, there exists a need for a system and method capable of plaque characteristics within patient vasculature. More particularly, there exists a need for intravascular ultrasound (IVUS) technology which will enable functional imaging approaches to assess plaque vulnerability.

### SUMMARY OF THE INVENTION

In one aspect of the present invention there is provided an intravascular ultrasound transducer device that is introduced into the blood vessel with a catheter, which utilizes contrast imaging to determine internal properties of the plaque such as the blood flow. The transducer device converts one form of energy to another so a small transducer will be used to send audio a catheter, the technology detects the amount and activity of the small blood vessels inside of the plaque which will illustrate the vulnerability of the plaque. The more of the small blood vessels, the more likely the plaque will be to grow larger or break away from the vessel wall and create a blood clot. The technology uses a contrast agent to enhance the contrast of structures or fluids within the human body, which allows the ultrasound machine to illuminate the blood vessel and any plaque present. The contrast agent must be activated and this technology utilizes a low audio frequency in the device. Once the contrast agent is activated, a higher audio frequency is generated and sends information back to the ultrasound machine. The higher frequency can provide an enhanced imaging of the small blood vessels in the plaque and any molecular makers of inflammation, and may have a significant impact on the estimation of the risk of plaque rupture and assessment of cardiovascular disease.

The technology allows ultrasound catheters to be used with contrast agents in a nonlinear imaging mode. One result is that the transducers have a higher sensitivity to blood flow, small blood vessel structures, and improved picture resolution. Nonlinear contrast agents, such as microbubbles, are used in ultrasound to enhance the deflection of radiation from blood. To increase contrast between these agents and tissue, nonlinear imaging methods, such as harmonic imaging or difference frequency imaging, can be used. For these, power is transmitted at one frequency and received at a different frequency. The intravascular ultrasound transducers can send a frequency low enough to activate the contrast agent (1-10 MHz) but, high enough to receive a higher frequency (above 20 MHz) to significantly increase the resolution of the imaging and illustrate the small blood vessels in plaque.

More particularly, technology is provided for atherosclerosis assessment with an IVUS catheter system for high-frequency contrast imaging, particularly for diagnostic approaches for vasa-vasorum imaging, specifically for pathologic neovascularization, and for IVUS-based molecular imaging of inflammation and other biomarkers of disease progression for improved clinical assessment of atherosclerotic plaques. Such contrast imaging techniques can provide critical information to assess plaque instability. However, nonlinear detection strategies for microbubble contrast agents are most effective near their resonant frequency, which is typically between 1-10 MHz, much lower that the IVUS imaging frequency (20-45 MHz). The high frequency imaging strategy utilizing the ultra-broadband response of contrast agents provides very high signal to noise, high-resolution contrast imaging at frequencies above 20 MHz, but requires a dual-frequency ultra-broadband transducer for IVUS using micromachined piezoelectric composite (MPC) ultrasound transducer technology, which may provide physicians with more accurate atherosclerosis diagnosis, dual-frequency ultra-broadband transducer for IVUS using micromachined piezoelectric composite (MPC) ultrasound transducer technology, which may provide physicians with more accurate atherosclerosis diagnosis, advance the understanding of the pathophysiology of coronary artery disease, and facilitate the development of novel cardiovascular drugs and device therapies.

In particular instances, PMN-PT and PIN-PMN-PT single crystal piezo-composite transducers with dual frequency capability (5 MHz and 35 MHz) may be formed using deep reactive ion etching and multilayer techniques. The 5 MHz transducer may be used for contrast agent excitation, and the 35 MHz piezo-composite transducers may be used as a receiver. The dual-frequency transducer may be mounted on a 3-French catheter.

These and other features, aspects, and advantages of the present invention will become better understood with reference to the following drawings, description, and claims.

### BRIEF DESCRIPTION OF THE DRAWINGS

FIGS. 1A-1E illustrate examples of contrast enhanced acoustic angiography of rodent microvasculature, with transmit 5 MHz/receive 30 MHz transducer device. FIGS. 1A and 1B are high resolution images of tortuous angiogenic microvasculature near a developing tumor in rats. FIGS. 1C and 1D are images of microvasculature from the same area on healthy animals as FIGS. 1A and 1B, showing normal microvasculature. Differences in healthy vs. angiogenic vasculature are readily apparent with acoustic angiography. Scale bar ˜5 mm; resolution is on the order of 100 microns. FIG. 1E is an image of a subcutaneous fibrosarcoma tumor in a rat with illustrating microvascular segmentation in red, enabled due to high SNR for microvessels, with 30 MHz tissue overlay in grayscale to provide anatomical reference. Scale bar for FIG. 1E is ˜7.5 mm. Images of the microvasculature with this resolution and signal-to-noise are possible because of the dual-frequency ultra-broadband technique disclosed herein;

FIGS. 2A and 2B illustrate 3-D molecular imaging with ultrasound and 2-D slices of an angiogenic tumor in a rat model. The presence of targeted contrast (green—FIGS. 2A and 2B) overlaid on anatomical b-mode images (gray—shown in FIG. 1B) is suggested to be correlated with spatial distribution of the angiogenesis biomarker aᵥb₃;

FIGS. 3A-3C illustrate scanning electron microscopy of an etched PMN-PT single crystal micro-array (3A), and top (3B) and bottom (3C) surfaces of PMN-PT/epoxy 1-3 composites;

FIGS. 4A and 4B illustrate impedance and phase spectrum of micromachined piezoelectric 1-3 composites at 40 MHz (4A) and 60 MHz (4B), with high electromechanical coupling coefficients for highly sensitive and broadband IVUS ultrasound transducers;

FIGS. 5A-5C illustrate a Boston Scientific® 3F IVUS catheter (5A) which is used as a host housing for the transducer element of the present disclosure; a diagram of rotational transducer head (5B); and an example of in-vivo IVUS image comparison (5C) using a commercial 40 MHz Boston Scientific transducer (5C—left) and a 60 MHz micromachined piezoelectric composite transducer (5C—right);

FIGS. 6A-6D illustrate modeling results of a receive impulse response of a 35 MHz piezo-composite transducer (0.5 mm×0.5 mm) (6A); the calculated pressure field (1-D) of a 0.6 mm×3 mm 5 MHz transducer under 200 V (6B); a 2D pressure field (3 mm lateral aperture) under 200 V (6C); and a 2-D pressure field (0.6 mm lateral aperture) under 200 V (6D);

FIG. 7 illustrates a schematic cross-sectional view of a dual frequency IVUS transducer assembly (5 MHz/35 MHz);

FIG. 8 illustrates an alternative dual frequency transducer assembly;

FIG. 9 is a schematic of dual frequency, contrast enhanced IVUS (CE-IVUS) using a circular/cylindrical transducer array;

FIG. 10 is a schematic of an 8 element 5 MHz and 64 element 40 MHz circular/cylindrical transducer array for CE-IVUS;

FIG. 11 is a schematic of a small-aperture 5 MHz/35 MHz dual frequency transducer;

FIGS. 12A and 12B schematically illustrate a performance characterization of a dual frequency transducer array as shown in FIG. 11, including transmission pressure at low frequency, and a pulse-echo result of the high frequency transducer (without matching);

FIGS. 13A and 13B schematically illustrate a simulation of a dual frequency transducer array as shown in FIG. 11, including a transmission field of the dual frequency transducer array, and receiving sensitivity of the dual frequency transducer array;

FIG. 14 schematically illustrates a two-channel imaging system implementing a dual frequency transducer array as shown in FIG. 11;

FIG. 15 schematically illustrates a testing arrangement for determining nonlinear responses of microbubbles; and

FIGS. 16A and 16B schematically illustrate spectrograms of data collected without and with microbubbles using a testing arrangement as shown in FIG. 15.

### DETAILED DESCRIPTION OF THE INVENTION

The present disclosure now will be described more fully hereinafter with reference to the accompanying drawings. The disclosure can be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

As previously discussed, cardiovascular disease is a major health problem, though mortality can be reduced with early intervention. Improved imaging methods, particularly for intravascular applications, to assess vulnerable plaques are critically needed. In this regard, it has been suggested that vasa vasorum neovasculature and inflammatory and angiogenic biomarkers are related to both plaque development and vulnerability. Acoustic angiography may have the potential to assess vasa vasorum neovasculature, but cannot be performed with current intravascular ultrasound (IVUS) systems. Ultrasound molecular imaging may have the potential to assess biomarkers of inflammation and angiogenesis, but cannot be performed on current IVUS systems.

As such, aspects of the present disclosure are directed to ultrasonic transducers, such as a dual-frequency ultra-broadband transducer for high contrast intravascular imaging, and catheter devices associated therewith. As disclosed, technology related to dual-frequency ultra broadband imaging may provide high signal-to-noise and high resolution contrast imaging, with enhanced penetration depth due to one way tissue attenuation, which may allow molecular imaging and acoustic angiography. In some instances, dual-frequency ultra broadband imaging can be implemented with IVUS catheters using, for example, photolithography-based micromachined piezo-composite transducers. In some aspects, some IVUS catheters may implement two broadband confocal transducer elements of spaced-apart, relatively low and relatively high frequencies. In one example, the high frequency transducer elements may have an effective operational frequency of greater than about 20 MHz, such as 35 MHz, 40 MHz, or greater. Further, the low frequency transducer elements may have an effective operational frequency of less than about 15 MHz, such as 5 MHz. Such a system may allow, for example, dual-frequency ultra-broadband imaging, producing relatively high resolution “acoustic angiography” for vasa vasorum imaging, as well as high signal to noise molecular imaging.

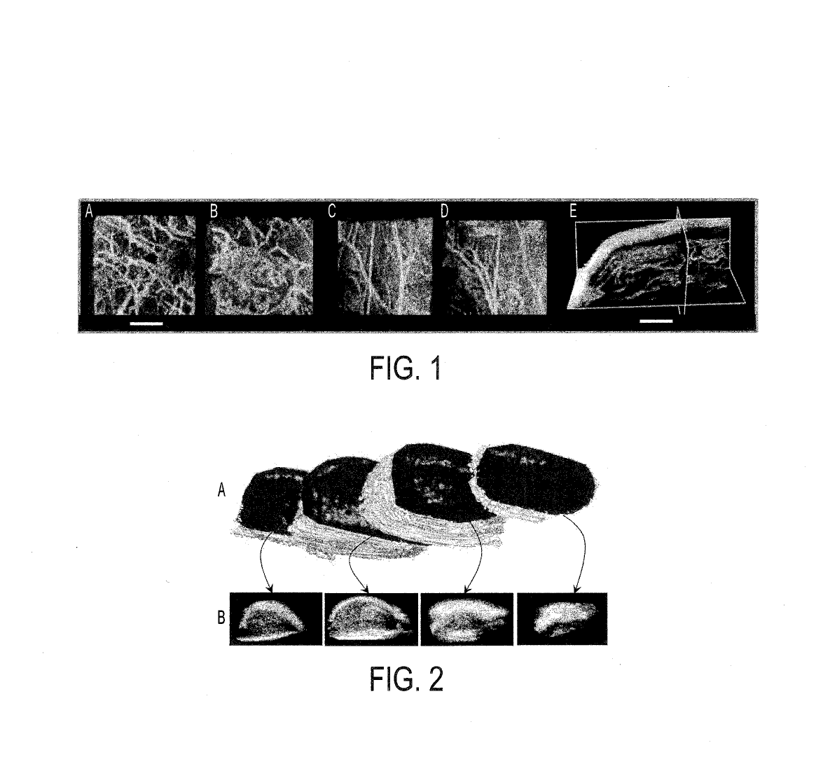

In one aspect, such a dual frequency transducer device may comprise a low frequency transducer element (e.g., about 4 MHz effective operational frequency) configured to transmit acoustic energy (i.e., in a transmit mode) or otherwise to excite microbubbles near resonance, and a high frequency transducer element (e.g., about 30 MHz effective operational frequency) configured to receive acoustic energy (i.e., in a receive mode) or otherwise to detect high-frequency energy from microbubbles only. That is such transducers can operate in a transmit 4 MHz/receive 30 MHz mode which detects only signals from microbubbles associated with a contrast agent (a high-pass filter may be used to remove all tissue components of the transducer signal), and also in a transmit/receive 30 MHz mode for anatomical reference. Such transducers may also be functional with a variety of commercially-available ultrasound platforms such as, for example, the Visualsonics Vevo commercial ultrasound system platform. Combining such transducers with the disclosed imaging strategy, in some instances, results in an enhanced contrast-to-tissue signal and resolution (FIGS. 1A-1D), with the capability of molecular imaging and acoustic angiography. Additionally, such technology may allow the display of contrast-only data on simultaneously-acquired b-mode tissue data (FIG. 1E). Thus, high-resolution, lowest-noise, contrast enhanced ultrasound images are achievable. In some aspects, 3-D ultrasound acoustic angiography may be performed, and segmentation and morphological analysis of microvessel structure, with high accuracy due to high signal to noise ratio in the ultrasound images, may also be conducted.

One aspect of the present disclosure may also involve the configuration and arrangement of both contrast agents and signal processing methods for molecular imaging with ultrasound. In this regard, microbubble contrast agents formulated with ligands such as echistatin, the antibody LM609, and cyclic RGD peptides may be utilized to target αᵥβ₃ integrin associated with angiogenesis (FIGS. 2A and 2B) or other targets of disease such as inflammation. Additionally, aspects of the present disclosure may be applicable to molecular imaging, which may be utilized to assess response to therapeutic treatment, and/or may be an indicator of tumor response prior to the point at which anatomical changes in the tumor become visible.

Another aspect of the present disclosure is directed to configurations of transducers capable of accomplishing enhanced contrast imaging, as otherwise disclosed herein. In this regard, composite piezoelectric materials have been known to demonstrate high electromechanical coupling factors, low acoustic impedance, and relatively ease of conformance. In some instances, though, appropriate diced feature sizes may be difficult to achieve when applied to high frequency transducers and, as such, the resulting transducer may have certain frequency limitations. Such frequency limitations may result, for example, from the frequency of the lateral mode resonance, which is determined by the shear wave velocity of the filler material and the width of the dicing cut. The frequency of the first lateral mode in a 1-3 composite can be empirically expressed as

$${f}_{1} = \frac{{V}_{T}}{2 \sqrt{2} {d}_{p}}$$

where f₁ is the frequency of the first lateral mode, V_{T} is the shear wave velocity of filler and dₚ is the kerf width. For example, if the kerf width is 10 μm, which may represent the current state of the art for dicing, the frequency of the first lateral mode is about 39 MHz, limiting the effective operating frequency of the resulting composite-based transducer device to about 20 MHz. Composites with a kerf width of about <7 μm and a volume fraction of between about 56% and about 70% may be required for 35 MHz (effective operating frequency) piezo-composite transducers.

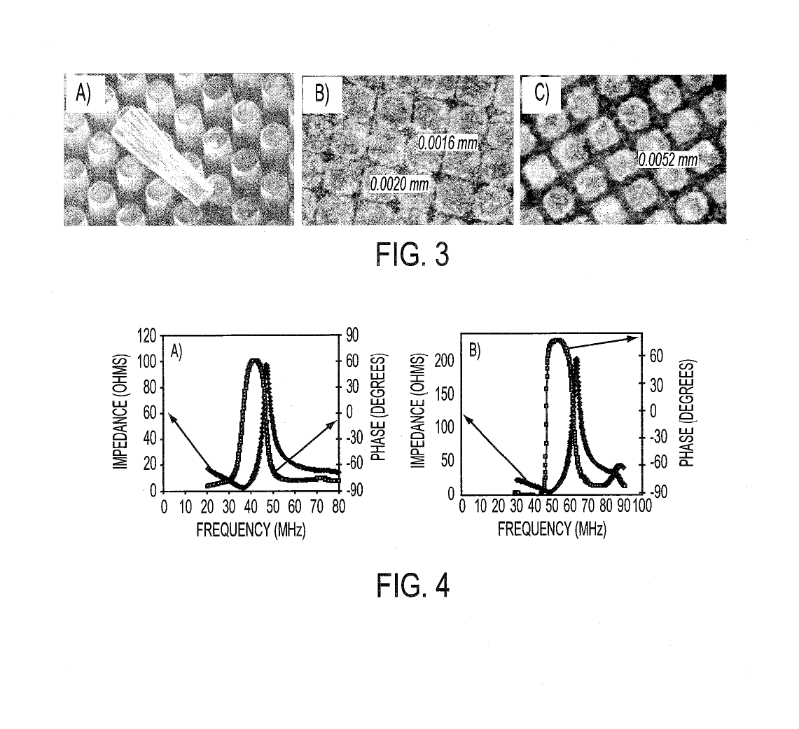

Fabrication processes for such high frequency transducers are preferably capable of processing single crystal piezoelectric materials, and capable of fine kerf processing. In this regard, some single crystal piezoelectric materials based, for example, on (1−x)Pb(Mg_{1/3}Nb_{2/3})O₃-xPbTiO₃ (PMN-PT), may demonstrate desirable performance advantages in high frequency transducers over conventional PZT ceramic transducer devices. In such instances, a photolithography-based deep dry etching of a PMN-PT single crystal may be suitable for high frequency 1-3 composite fabrication (FIGS. 3A-3C) of suitable transducer elements. Photolithography-based micromachining may have some advantages compared with conventional ultrasound transducer and transducer array fabrication techniques, including, for example, submicron machining precision, batch fabrication, a low-stress mechanical environment for fragile, fine structures, and the possibility for integrated array design. FIG. 3A shows a SEM picture of etched PMN-PT posts with width of ˜15 μm and the kerf size of ˜6 μm. Such kerfs may, for instance, be then filled with epoxy, followed by precision lapping and forming 1-3 composites (FIGS. 3B and 3C). The resulting transducer devices may thus demonstrate 40 MHz and 60 MHz effective operating frequency with such micromachined single crystal 1-3 composites, in addition to desirable electromechanical coupling coefficients (i.e., >0.75 at frequencies >40 MHz) (FIGS. 4A and 4B), thereby providing highly sensitive and broadband IVUS ultrasound transducers.

That is, such 1-3 PMN-PT composite (40 MHz and 60 MHz) IVUS transducers may more favorably compare with conventional ceramic material PZT-5H (40 MHz) transducers, demonstrating, for example, higher bandwidth and sensitivity (Table 1).

TABLE 1
IVUS transducer comparison

| | | | Sensitivity |
|---|---|---|---|
| HF Transducers | Center frequency | Bandwidth (−6 dB) | (p-p) |
| Commercial 40 | 39.9 MHz | 43.5% | 154 mV |
| MHz | | | |
| 40 MHz | 40.9 MHz | 76.5% | 191 mV |
| piezo-composite | | | |
| transducer | | | |
| 60 MHz | 55 MHz | 77% | ~200 mV |
| Piezo-composite | | | |
| transducer | | | |

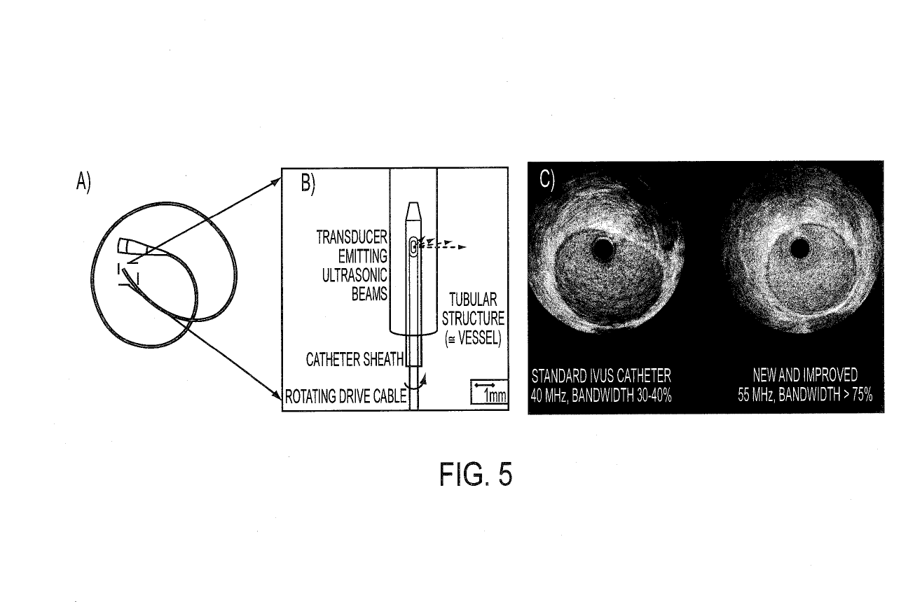

According to some aspects of the disclosure, such piezo-composite transducer elements may be adapted to be engaged with a catheter (hollow lumen) for catheter imaging purposes. For example, such a transducer device may be mounted on a 3-French catheter, for example, for pre-clinical animal studies (FIGS. 5A and 5B). FIG. 5C shows a comparison of IVUS images obtained using a 40 MHz standard transducer and a 60 MHz piezo-composite transducer, respectively. Such images and image loops show a significantly finer blood speckle for the piezo-composite transducer at 60 MHz than a standard transducer at 40 MHz, and, in addition, provide visual distinction of vessel structures such as the coronary intima and stent struts. In some instances, such piezo-composite transducer may demonstrate a broad or broader bandwidth, and may provide, for instance, clear thrombus delineation in such IVUS imaging using a multi frequency signal ratio method, thereby indicating that high-frequency piezo-composite transducers can be excellent receivers for IVUS contrast imaging.

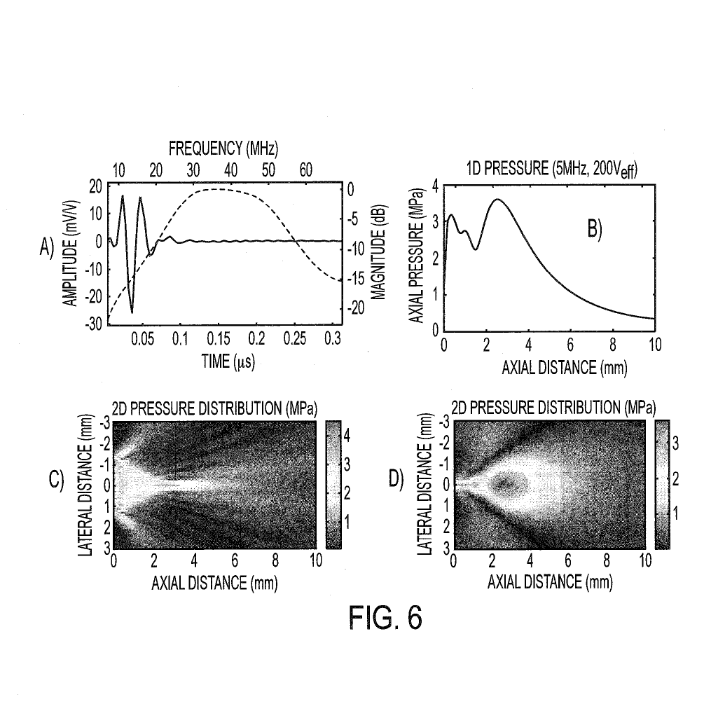

In one aspect, the transducer device may comprise a 3-layer 5 MHz (LF) transducer with an aperture of <0.6 mm×3 mm (0.6 mm in the radial direction and 3 mm along the axial direction of a 3-French catheter), as a transmitter for IVUS contrast imaging, with a pressure field of amplitude >2 MPa for bubble excitation. The transducer device may further comprise a 35 MHz (HF) piezo-composite transducer with −6 dB bandwidth >80%, as a receiver, and integrated with the 5 MHz transducer (transmitter) for dual-frequency IVUS contrast imaging. In one arrangement, the HF transducer may be embedded in the LF transducer, wherein parameters used in a Krimholtz, Leedom, and Matthaei (KLM) model and beam profile modeling are shown in Table 2. Simulations (FIGS. 6A-6D) indicate that the HF piezo-composite transducers may have a −6 dB bandwidth of ˜80% (FIG. 6A), and that the 1-layer LF transducer (transmitter) may produce a peak pressure of >3.5 MPa under a driving voltage of 200 Vₚ₋ₚ (FIG. 6B-D). In some instances, a lower driving voltage (e.g., <70 Vₚ₋ₚ) may be required for a 3-layer LF transducer to generate a similar or higher pressure field.

TABLE 2
Initial dual frequency transducer design.

| Center | | Active | | |
|---|---|---|---|---|
| Frequency | Aperture | thickness | Matching | Backing |
| 35 MHz | 0.5 mm × | 0.039 mm | 7 MRayl/0.014 mm | 18 MRayl/ |
| | 0.5 mm | | | 0.43 mm |
| 5 MHz | 0.6 mm × | 0.36 mm | 18 MRayl/0.039 mm + | 6 MRayl/ |
| | 3 mm | | 7 MRayl/0.014 mm | |

In this regard, particular aspects of the transducer device may be varied, which may affect the performance thereof, wherein such variations may include, for example, a multilayer transmitter (e.g., a 3-layer 5 MHz transducer) and/or more matching layer variations (i.e., for broader bandwidth and higher sensitivity).

With regard to the multilayering aspect, multilayering is a technique in the piezoelectrics art that provides increases in signal-to-noise ratio through better electrical matching between transducer elements and the associated electronics. For IVUS contrast imaging, this technique may improve power transfer efficiency of the transducer in transmit mode. Multilayer transducers may be capable of increasing capacitance thereof by a factor of N² since the layers are stacked mechanically in series and electrically connected in parallel (N-layer number). In the transducer stack, the total thickness is maintained, and therefore each layer thickness is decreased by a factor of N. As such, in transmit mode, the power output Pₒᵤₜ=Vₒᵤₜ²/Rₘ may be maximized when the mechanical resistance is minimized. Given

$${R}_{m} = \frac{π}{4 {k}_{eff}^{2} ω {C}_{0}} \cdot {Z}_{a}$$

where k_{eff} is the electromechanical coupling of the piezoelectric and Zₐ is the ratio of front acoustic loads to that of the piezoelectric, in a multilayer transducer, the Rₘ is decreased by a factor of N², resulting in an equal increase in power output.

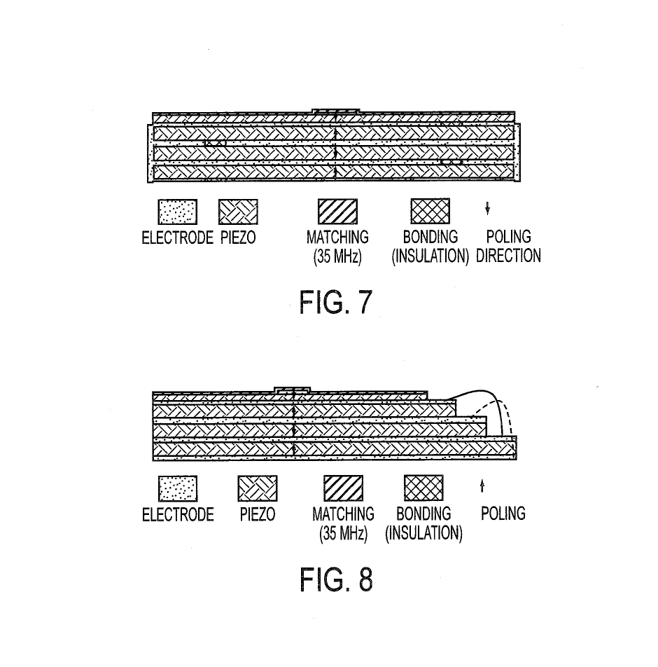

In some instances, lead magnesium niobate-lead titanate (PMN-PT) and lead indium niobate-lead magnesium niobate-lead titanate (PIN-PMN-PT) may be used to form such single crystal multilayer single element transducers for dual frequency IVUS contrast imaging, as disclosed herein. PMN-PT single crystals have been demonstrated for piezo-composite transducers in the frequency range of 15-75 MHz. PIN-PMN-PT single crystals may also be used for dual frequency piezo-composite transducers, and may provide an enhanced coercive field (which may be important to achieve the pressure fields required for contrast imaging). The fabrication of 35 MHz piezo-composite transducer receivers may follow a standard piezo-composite microfabrication process. For the 3-layer 5 MHz transmitter, 1-3 piezo-composite layers with wrap-around electrodes may be fabricated using a dice-and-fill technique, followed by multilayer bonding and attachment of the 35 MHz piezo-composite layer and matching layers (FIG. 7), to produce the dual frequency transducer, as disclosed herein.

Performance of the dual frequency transducer device can be assessed in various manners. For example, pulse-echo experiments and raster scanning with a calibrated hydrophone may be performed in a water tank to obtain sensitivity, beam profile, and power output of the LF and HF transducer components. Further, performance of the transducer system under various imaging parameters, such as driving amplitude, pulse length, and driving frequency, may be assessed using, for instance, a Tektronix Arbitrary Waveform Generator (AWG2021) and RF Amplifier (ENI 3100LA), along with a diplexor unit and receiver (Ritec BR640), and a Signatec 400 MHz 14 bit A-D card (PX14400).

In one aspect, the dual frequency transducer device, as disclosed herein may be mounted in the distal portion or to the distal tip of a 3-French catheter using a rectangular housing first attached to the catheter shaft using a suitable adhesive such as UV epoxy. The transducer device may be potted into the housing and/or secured with UV epoxy. The ground wire may be attached to the backing layer of the LF transducer using conductive epoxy, and the signal wire of the LF transducer may also function as the ground wire of the HF transducer.

In some instances, the multilayer transducers may implement a “wrap-around” electrode, wherein an electrode isolation strip may be diced on each side of the electrode composite plates (FIG. 7) prior to application of the electrode. Alternatively, a pyramid type of structure (FIG. 8) may be implemented, wherein two interconnect wires may be bonded inside the catheter.

In some aspects, microbubble contrast agents may be implemented to allow acquisition of contrast-enhanced ultrasound images. In such instances, the low-frequency excitation element (LF transducer in transmit mode) may be driven with a Tektronix Arbitrary Waveform Generator (AWG2021) and RF Amplifier (ENI 3100LA). Contrast echoes may be acquired via the HF receiver transducer with a Ritec receiver (BR640) and Signatec 400 MHz 14 bit A-D card (PX14400). A seventh-order high-pass filter (10, 15, or 20 MHz cutoffs—TTE Inc.) may be utilized to retain only high-frequency broadband energy from bubble harmonics. The RF pulser/receiver system may be synchronized with the trigger signal from catheter rotation. A Boston Scientific Galaxy IVUS system motor drive may be utilized to permit acquisition of a series of 2-D images. The catheter may also be manually moved along the axis thereof with a precision micrometer stage to build a 3-D data set. Imaging metrics that may be include contrast-to-tissue ratio as a function of depth, resolution, and sensitivity to contrast agent dose circulating in a simulated vasa vasorum.

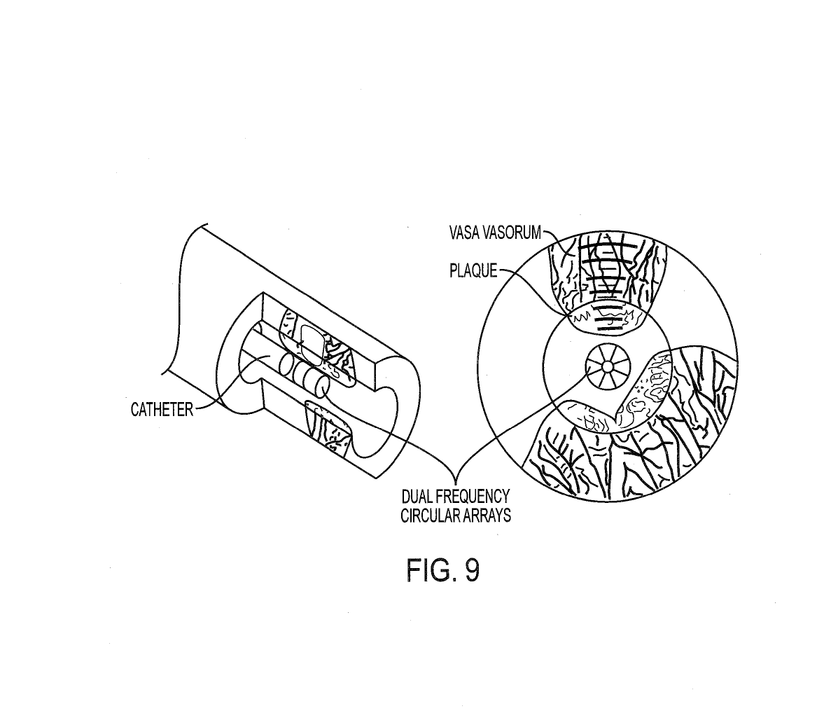

In other aspects, micromachined multilayer piezo-composite transducer technology may use a through-wafer-via technique to fabricate a 3-layer 8-element piezo-composite transmitting transducer array with an element aperture of 0.5 mm×3 mm, and a single layer 40 MHz 64-element MPC circular array may be integrated as a receiver. Significantly reduced electrical impedance due to the multilayer technique will allow sufficient acoustic power transmission from a low frequency, small aperture 5 MHz transducer (e.g., >2 MPa), while the broadband high frequency MPC transducer will ensure high resolution contrast imaging. Moreover, no mechanical rotation is needed for one aspect of the dual frequency phased array based CE-IVUS. In addition to the function of CE-IVUS, the 40 MHz circular or cylindrical array itself can be used for conventional IVUS for lumen and vessel wall profile imaging with improved resolution compared to 20 MHz counterparts. The resulting dual frequency CE-IVUS catheter (FIG. 9) may give IVUS the capability to provide additional information about vasa vasorum microvascular structure and molecular changes associated with angiogenesis, inflammation, and thrombus, in order to allow better assessment of atherosclerotic lesions.

A dual frequency circular/cylindrical transducer array may also be provided in one aspect. Low frequency: A multilayer, 5 MHZ 8-element circular/cylindrical transducer array with single element aperture of <0.5 mm×3 mm (0.5 mm in radial direction and 3 mm along the axial direction of catheter) may be provided as a transmitter for IVUS contrast imaging. The pressure field with amplitude >2 MPa at the 3 mm focal point is implemented for driving microbubbles effectively at depth to produce broadband energy. High frequency: A 40 MHz 64-element MPC circular/tubular array with −6 dB bandwidth >90% may be implemented as the high frequency transducer array and integrated with the low frequency transmitter for IVUS contrast imaging. This dual frequency transducer will be able to transmit at 5 MHz and receive with the 40 MHz array for contrast imaging, and can be used in traditional 40 MHz pulse echo for B-mode for anatomical imaging with improved resolution compared to its 20 MHz IVUS phased array counterpart.

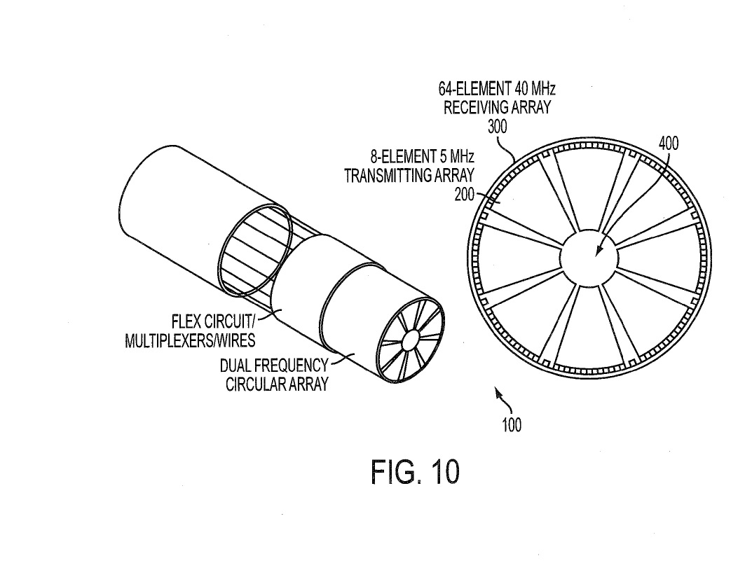

More particularly, a dual frequency circular/cylindrical array 100 is shown in FIG. 10, and may be implemented within a distal end of a catheter (or “hollow lumen”). The 8-element 5 MHz circular/cylindrical transducer array 200 may be used to transmit acoustic waves into coronary tissue wall, and the 64-element 40 MHz circular/tubular transducer array 300 may be used to receive nonlinear response from microbubbles when excited at 5 MHz. The overall array 100 can also be used in pulse-echo mode at 40 MHz for high resolution grayscale IVUS imaging. An inner diameter 400 of >0.5 mm allows for passage of the guide wire through the catheter. The outer diameter of <2 mm is standard for coronary catheter application. Particular exemplary specifications are given in Table 3.

TABLE 3
Initial design specifications.

| | Low frequency | High frequency |
|---|---|---|
| | array | array |
| Center frequency | 5-10 MHz | 40 MHz |
| Element number | 8 | 64 |
| Pitch | 0.584 mm | 0.073 mm |
| Transmitting | >2 MPa at <100 | 30 kPa/1 Vₚ₋ₚ |
| pressure | Vₚ₋ₚ | |
| Receiving | n/a | >100 nV/Pa |
| sensitivity | (transmit only) | |
| Bandwidth (−6 dB) | — | >80% |
| Side lobe | — | <−30 dB |
| amplitude | | |

The transducer performance may be optimized for contrast IVUS imaging using: 1) multilayer transmitter (e.g. 3-layer 5 MHz transducer) modeling; 2) more matching layer designs for broader bandwidth and higher sensitivity; 3) 3D pressure field modeling; and 4) receiving beam profile modeling.

Electrical impedance spectra, transmitting impulse and pulse-echo response may be evaluated to determine the transmitting sensitivity and receiving bandwidth of the transducer device. The initial matching layer configuration may provide a high frequency receiving bandwidth >100%. Variations in high frequency matching layers and low frequency matching layers may be considered so that bandwidth >90% for the 40 MHz array and transmitting pressure field >2 MPa for the 5 MHz array may be obtained. Azimuthal directivity of a single element may be calculated to provide a baseline for the aperture with no crosstalk. Beam steering may also be evaluated using multiple receiving elements to determine the effect of the designed pitch.

### Example

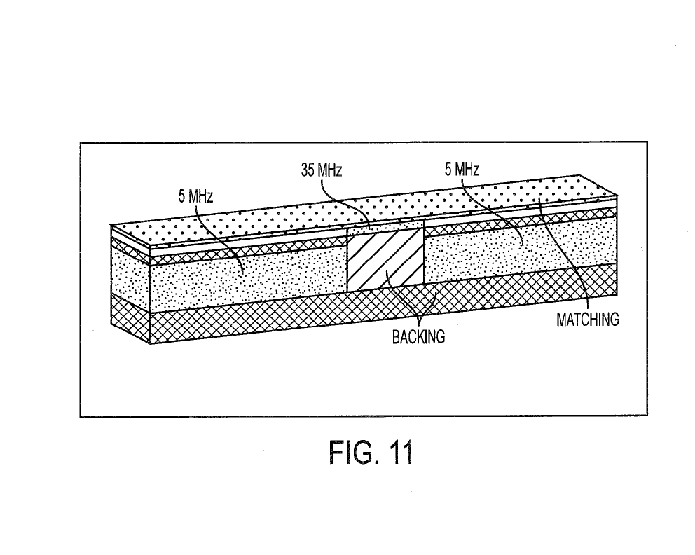

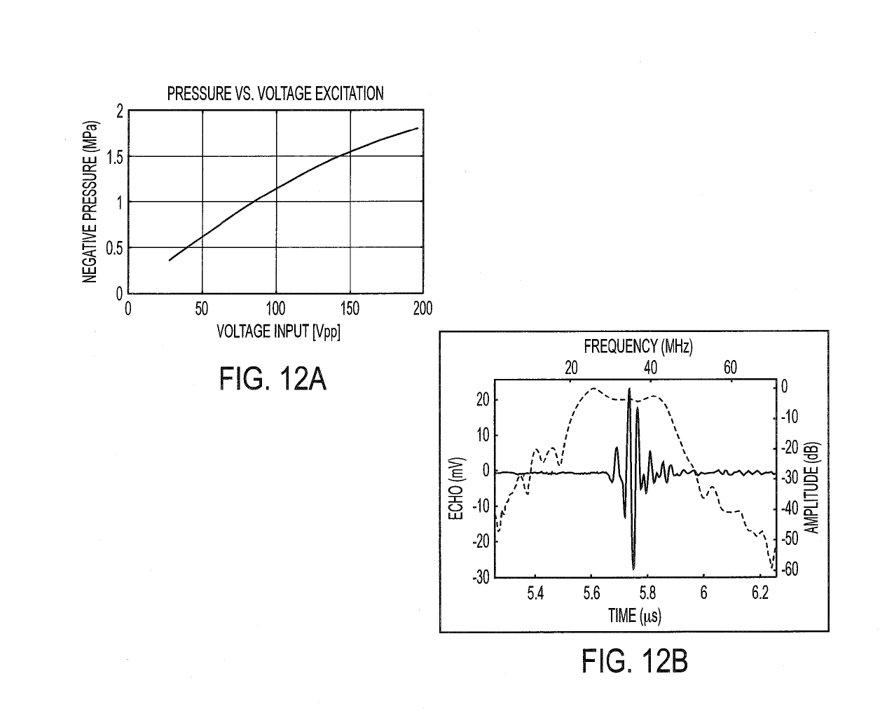

Since one aspect of the dual frequency IVUS array may be assembled into 3-French catheters, small aperture transducers may be required. Small aperture 5 MHz, 35 MHz and dual frequency 5 MHz/35 MHz were developed. The low frequency (5 MHz) transmitting transducer with aperture of 0.6 mm×3 mm was implemented to excite microbubbles in micro-vessels, while the high frequency receiver (35 MHz) with aperture of 0.6 mm×0.6 mm was used to receive nonlinear echoes from microbubbles for high resolution imaging. FIG. 11 shows a schematic view of such a dual frequency IVUS transducer. Such a dual frequency transducer may be fabricated and housed on the tip of a 20 gauge thin-walled needle, and a transmission pressure of the transducer may be determined. The center frequency was measured to be 6.5 MHz, instead of the specified 5 MHz, because a thinner piezo plate was implemented. The negative pressure was found to be 1.8 MPa with a 5-cycle burst excitation at 200 Vₚ₋ₚ (see, e.g., FIG. 12A). The higher frequency probe peaked at MI=0.60. However, a slight lower MI could have been used since nonlinear microbubble response thresholds at high frequencies were not calculated. FIG. 12B shows the impulse response of the receiving transducer. The −6 dB fractional bandwidth of the receiving transducer was about 60% and the loop sensitivity was about −35 dB, comparing favorably with particular commercial IVUS transducers.

The dual frequency transducer array consisted of the transmission array and the receiving array. There were 8 elements in the transmission array, forming a cylinder with diameter of approximately 1 mm. At the outer surface of the transmission array, 64 high frequency elements formed the receiving array (see, e.g., FIG. 10). Table 4, below, summarizes some parameters of the dual frequency array for acoustic field simulations:

TABLE 4
Parameters of the dual frequency transducer array

| IVUS dual | | |
|---|---|---|
| frequency array | 5 MHz | 40 MHz |
| piezoelectric material | PMN-PT 1-3 composite | PMN-PT 1-3 composite |
| matching material | PMN-PT 1-3 composite | Parylene |
| | and parylene | |
| backing material | silver epoxy | PMN-PT 1-3 composite |
| | | and silver epoxy |
| element number | 8 | 64 |
| element width (μm) | 584 | 50 |
| kerf (μm) | 84 | 13 |
| element length (mm) | 3 | 3 |

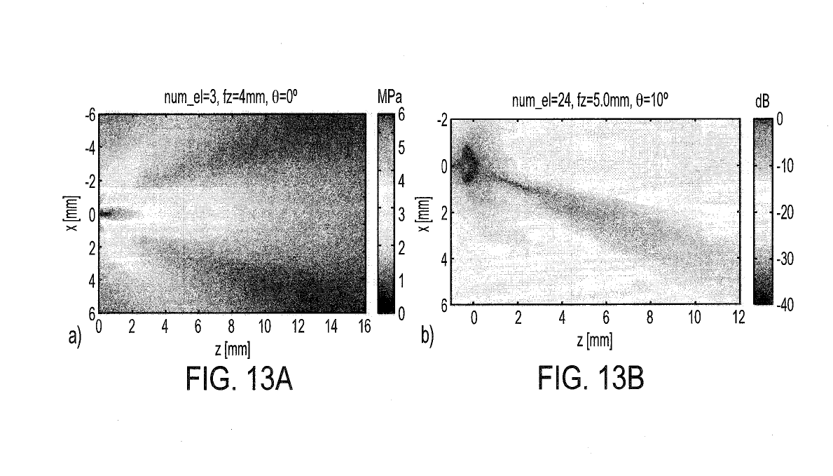

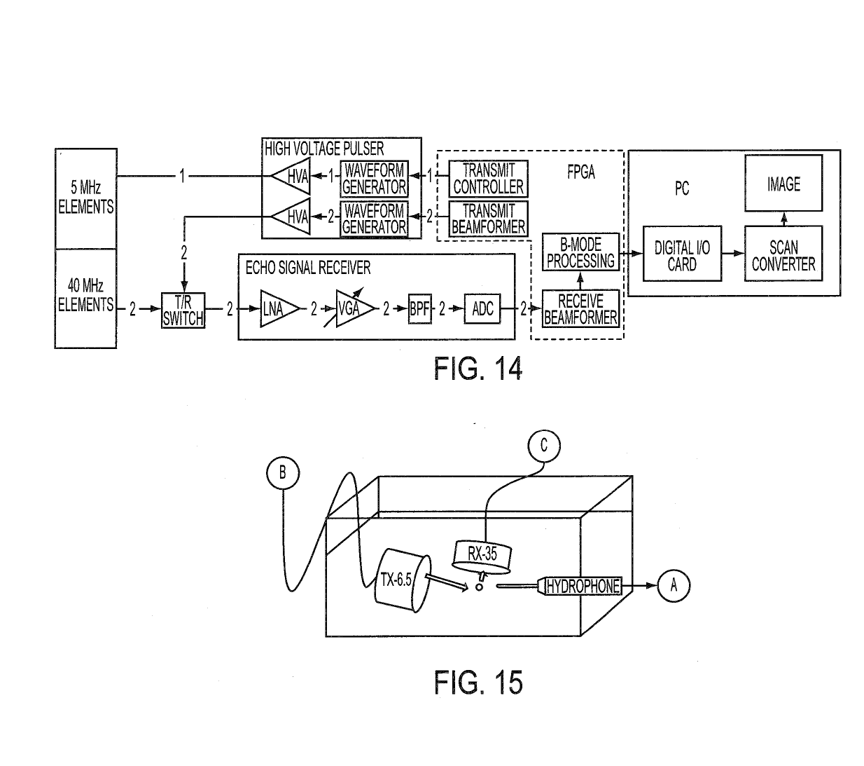

A field map of the transmission and receiving array was simulated with Field II toolkit based on MATLAB. In one instance, it was found that 3 elements of the transmission array and 24 elements of the receiving array yielded sufficient transmitting pressure and receiving sensitivity, as shown, for example, in FIGS. 13A and 13B. A dual frequency 5 MHz/40 MHz imaging system was formed and tested in a two-channel system using hardware as shown, for example, in FIG. 14. Such a two-channel system may include, for instance, hardware and/or software for two independent pulser and TX/RX switches, one for a transducer having a center frequency of about 40 MHz and the other for a transducer having a center frequency of about 5 MHz. The two-channel system may also include hardware/software for signal pre-amplification, digitalization, beamforming and central control, and data communication. In some instances, the imaging system may include dual TX/RX modes, with one mode transmitting on one of the eight 5 MHz array elements and receiving from a group of 40 MHz array elements from the 64-element high frequency array to form contrast images, with the other mode transmitting on some of the 40 MHz array elements and receiving from some 40 MHz array elements to form synthetic B-mode images.

Contrast agent response can be tested with two small-aperture single-frequency transducers in a degassed water bath, as shown, for example, in FIG. 15, with the transmitter having an aperture of about 0.6 mm×3 mm and a center frequency of about 6.5 MHz, and the receiver having an aperture of about 0.6 mm×0.6 mm and a center frequency of about 35 MHz. The two transducers may be aligned, for instance, using mechanical and/or computer-controlled 3-axis motion stages to register the foci of the transducers to a particular region of interest, for example, a small (200 μm diameter) cellulose tube where microbubbles could be allowed to flow. A needle hydrophone can be used to measure the pressure signal from a transmitter source.

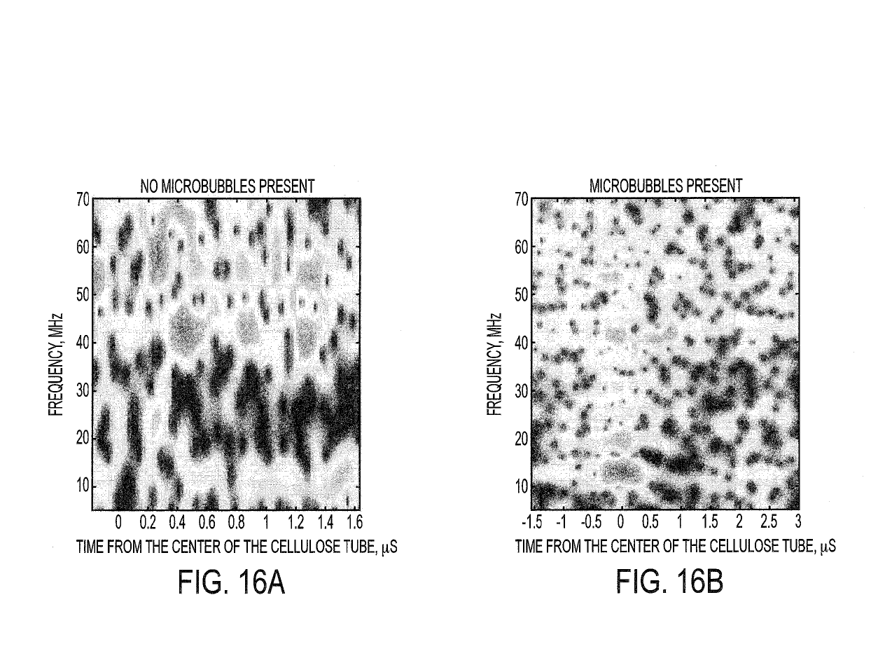

Non-linear echoes produced in the spatial location of microbubbles may be detected using two separate single-frequency IVUS probes. Microbubbles excited with increasing pressures were used to determine a threshold for signal detection (i.e., −0.95 MPa, 5 cycle burst, pulse repetition frequency=20 Hz, f_{c}=6.5 MHz). In this regard, FIGS. 16A and 16B illustrate respective spectrograms of collected data, one with microbubbles present in the tube (FIG. 16B) and another without microbubbles (FIG. 16A). In some instances, nonlinearly oscillating microbubbles have been shown to have frequency components at or near integer multiplies of the frequency at which they are excited, as shown, for instance, in the spectrogram of FIG. 16B in relation to frequency peaks (shown in red) at 13 MHz, 19.5 MHz, etc., when microbubbles are present.

Accordingly, as disclosed herein, in one aspect, an imaging catheter system may be provided, comprising a hollow lumen having a distal portion. A plurality of first transducer elements may be arranged in a first array configured to be received within the distal portion of the hollow lumen, with each of the first transducer elements comprising a micromachined piezo-composite, and the plurality of first transducer elements being configured to operate at an effective operational frequency of greater than about 30 MHz. In some instances, the plurality of first transducer elements may be configured to operate at an effective operational frequency of about 40 MHz.

In some aspects, the first array may be substantially planar, and configured to be received within the distal portion of the hollow lumen, such that the plane of the first array is substantially parallel to a longitudinal axis of the hollow lumen. A rotator device may be operably engaged with the first array, and configured to rotate the first array within the hollow lumen and about the longitudinal axis thereof. In some instances, a plurality of second transducer elements may be arranged in a second array, with each of the second transducer elements comprising a micromachined piezo-composite, and the second array being substantially planar. The plurality of second transducer elements may be configured to operate at an effective operational frequency of less than about 15 MHz. In some instances, the plurality of second transducer elements may be configured to operate at an effective operational frequency of about 5 MHz.

In some aspects, the first and second arrays may be arranged to be engaged such that the planes of the first and second arrays are substantially parallel and such that the respective first and second transducer elements define a single combined array. Further, the first and second transducer elements of the combined array may be confocally arranged.

The second transducer elements may be configured to be operated in one of a transmit mode and a receive mode, and the first transducer elements may be configured to be operated in the other of the transmit mode and the receive mode so as to facilitate contrast imaging.

The hollow lumen having the engaged transducer assemblies disposed within the distal portion thereof may, in some instances, be configured for intravascular application.

In some aspects, the first transducer elements forming the first array may be further arranged to define a tubular transducer assembly. In some instances, a plurality of second transducer elements may be arranged in a second array, with each of the second transducer elements comprising a micromachined piezo-composite, and the plurality of second transducer elements forming the second array being further arranged to define a cylindrical transducer assembly. The plurality of second transducer elements may be configured to operate at an effective operational frequency of less than about 15 MHz. In some aspects, the plurality of second transducer elements may be configured to operate at an effective operational frequency of about 5 MHz.

In some aspects, the cylindrical transducer assembly may be configured to be disposed within the tubular transducer assembly such that the first and second arrays are concentrically arranged. In other aspects, the first and second arrays may be concentrically arranged such that the respective first and second transducer elements define a single combined array. Further, the first and second transducer elements of the combined array may be confocally arranged.

The second transducer elements may be configured to be operated in one of a transmit mode and a receive mode, and the first transducer elements may be configured to be operated in the other of the transmit mode and the receive mode so as to facilitate contrast imaging.

In some aspects, the hollow lumen having the concentrically-arranged transducer assemblies disposed within the distal portion thereof may be configured for intravascular application.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. For example, intravascular ultrasound technology, as disclosed herein, may allow enhanced imaging of adventitial neovasculature, as well as molecular markers of inflammation, and has the potential to have an impact in the estimation of the risk of plaque rupture and assessment of atherosclerotic cardiovascular disease. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An imaging catheter system, comprising:
a hollow lumen having a distal portion; and
a plurality of first transducer elements arranged in a first array configured to be received within the distal portion of the hollow lumen, each of the first transducer elements comprising a micromachined piezo-composite, and the plurality of first transducer elements being configured to operate at an effective operational frequency of greater than about 30 MHz.

2. A system according to claim 1, wherein the plurality of first transducer elements is configured to operate at an effective operational frequency of about 40 MHz.

3. A system according to claim 1, wherein the first array is substantially planar, and is configured to be received within the distal portion of the hollow lumen such that the plane of the first array is substantially parallel to a longitudinal axis of the hollow lumen.

4. A system according to claim 3, further comprising a rotator device operably engaged with the first array, and configured to rotate the first array within the hollow lumen and about the longitudinal axis thereof.

5. A system according to claim 3, further comprising a plurality of second transducer elements arranged in a second array, each of the second transducer elements comprising a micromachined piezo-composite, and the second array being substantially planar.

6. A system according to claim 5, wherein the plurality of second transducer elements is configured to operate at an effective operational frequency of less than about 15 MHz.

7. A system according to claim 5, wherein the plurality of second transducer elements is configured to operate at an effective operational frequency of about 5 MHz.

8. A system according to claim 5, wherein the first and second arrays are arranged to be engaged such that the planes of the first and second arrays are substantially parallel and such that the respective first and second transducer elements define a single combined array.

9. A system according to claim 8, wherein the first and second transducer elements of the combined array are confocally arranged.

10. A system according to claim 8, wherein the second transducer elements are configured to be operated in one of a transmit mode and a receive mode, and the first transducer elements are configured to be operated in the other of the transmit mode and the receive mode so as to facilitate contrast imaging.

11. A system according to claim 8, wherein the hollow lumen having the engaged transducer assemblies disposed within the distal portion thereof is configured for intravascular application.

12. A device according to claim 1, wherein the first transducer elements forming the first array are further arranged to define a tubular transducer assembly.

13. A device according to claim 12, further comprising a plurality of second transducer elements arranged in a second array, each of the second transducer elements comprising a micromachined piezo-composite, and the plurality of second transducer elements forming the second array being further arranged to define a cylindrical transducer assembly.

14. A device according to claim 12, wherein the plurality of second transducer elements is configured to operate at an effective operational frequency of less than about 15 MHz.

15. A device according to claim 12, wherein the plurality of second transducer elements is configured to operate at an effective operational frequency of about 5 MHz.

16. A device according to claim 12, wherein the cylindrical transducer assembly is configured to be disposed within the tubular transducer assembly such that the first and second arrays are concentrically arranged.

17. A device according to claim 16, wherein the first and second arrays are concentrically arranged such that the respective first and second transducer elements define a single combined array.

18. A system according to claim 17, wherein the first and second transducer elements of the combined array are confocally arranged.

19. A device according to claim 17, wherein the second transducer elements are configured to be operated in one of a transmit mode and a receive mode, and the first transducer elements are configured to be operated in the other of the transmit mode and the receive mode so as to facilitate contrast imaging.

20. A device according to claim 17, wherein the hollow lumen having the concentrically-arranged transducer assemblies disposed within the distal portion thereof is configured for intravascular application.
